(19) United States
(12) Patent Application Publication
(10) Pub. No.: US 2012/0304995 A1
(43) Pub. Date: Dec. 6, 2012

(54) Previous Set Up Mode Parameter Retention

(75) Inventor: Richard Kauc, Milton (CA)

(73) Assignee: Nellcor Puritan Bennett LLC, Boulder, CO (US)

(21) Appl. No.: 13/149,722

(22) Filed: May 31, 2011

(51) Int. Cl. A61M 16/00; G06F 3/048

(52) U.S. Cl. 128/204.23; 128/204.21; 715/781

(57) ABSTRACT

The present application describes a previous mode button that allows a clinician to select any previously administered ventilation mode for a patient such that the previously input ventilation parameters for the selected ventilation mode will be used during ventilation. Upon receiving the selection of the previous mode button, a first stored previous mode and first ventilation parameters associated with the first stored previous mode are displayed. A determination is then made as to whether the displayed first stored previous mode is an appropriate mode. If the displayed first stored previous mode is an appropriate mode, ventilation is administered using the first stored previous mode and first ventilation parameters. If a determination is made that the first stored previous mode is not appropriate, a second stored previous mode is retrieved and displayed.

### INTRODUCTION

Inputting ventilation parameters for a ventilation mode can be a time consuming process. Oftentimes, ventilation parameters need to undergo a trial and error process before the appropriate parameters are set for a ventilation mode. If the condition of the patient changes, a clinician may need to select a new ventilation mode by which the patient will be ventilated. When a new ventilation mode is selected, the clinician needs to select new ventilation parameters appropriate for the new ventilation mode. Previously, ventilators have been equipped with a previous mode button. The previous mode button allows the ventilator to save ventilation parameters for the previously administered ventilation mode. Upon selection of the previous mode button, the ventilator delivers breaths in the previously administered ventilation mode using the previously stored ventilation parameters. However, the previous mode button only stores ventilation parameters associated with the last administered ventilation mode, thus only storing parameters for one previously administered ventilation mode. During ventilation, if a patient's condition changes, requiring more than two ventilation modes, the clinician will be forced to re-input ventilation parameters for any ventilation mode other than the last administered ventilation mode.

### Previous Set Up Mode Parameter Retention

The present application describes a previous mode button that allows a clinician to identify and select any previously administered ventilation mode for a patient such that the previously input ventilation parameters for the selected ventilation mode will be used during ventilation. In one embodiment, systems and methods are described for administering ventilation by a mechanical ventilator using a previously stored ventilation mode. A selection of a previous mode button is received. Upon receiving the selection of the previous mode button, a first stored previous mode and first ventilation parameters associated with the first stored previous mode are displayed. A determination is then made as to whether the displayed first stored previous mode is an appropriate mode. If the displayed first stored previous mode is an appropriate mode, ventilation is administered using the first stored previous mode and first ventilation parameters. If a determination is made that the first stored previous mode is not appropriate, a second stored previous mode is retrieved. The second stored previous mode and second ventilation parameters associated with the second stored previous mode are then displayed. A determination is then made as to whether the second stored previous mode is an appropriate mode. If not, a third stored previous mode and associated third ventilation parameters may be retrieved. In this manner, a clinician may select which mode, out of any previously administered mode, is appropriate for ventilation and ventilate using the appropriate mode and its associated ventilation parameters.

In another embodiment, a graphical user interface for administering ventilation by a mechanical ventilator using a previously stored ventilation mode is described. The graphical user interface may include at least one window associated with the graphical user interface and one or more elements within the at least one window. The one or more elements may further comprise a previous mode button. The previous mode button allows for the selection of any one ventilation mode of the previously administered ventilation modes and the ventilation parameters associated with the one ventilation mode.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of described technology and are not meant to limit the scope of the invention as claimed in any manner, which scope shall be based on the claims appended hereto.

FIG. 1 is a diagram illustrating an embodiment of a ventilator connected to a human patient.

FIG. 2 is a block-diagram illustrating an embodiment of a ventilatory system having a user interface for operating a ventilator using previously stored parameters for one or more ventilation modes.

FIG. 3 is an illustrative flowchart for storing parameters for multiple ventilation modes.

FIG. 4 is an illustrative flowchart for selecting a ventilation mode with previously stored parameters.

FIG. 5 is an illustration of a user interface for ventilation using ventilation modes with previously stored parameters.

### DETAILED DESCRIPTION

For the purposes of this disclosure, a “breath” refers to single cycle of inspiration and exhalation delivered with the assistance of a ventilator. The term “breath type” refers to some specific definition or set of rules dictating how the pressure and flow of respiratory gas is controlled by the ventilator during a breath. Breath types may be mandatory breath types (that is, the initiation and termination of the breath is made by the ventilator) or spontaneous breath types (which refers to breath types in which the breath is initiated and terminated by the patient).

Breath types may also be separated into pressure breath types and volume breath types. In general, pressure breath types deliver a target pressure at the patient airway during inhalation. Exemplary pressure breath types may include Pressure Control (PC) breath type, Pressure Support (PS) breath type, Continuous Positive Airway Pressure (CPAP) breath type, Volume Control Plus (VC+) breath type, Volume Support (VS) breath type, Proportional Assist (PA) breath type, and Tube Compensation ETC) breath type. Alternatively, volume breath types are set to deliver a clinician-selected peak flow and flow patter to achieve a clinician-selected tidal volume. An exemplary volume breath type may be a Volume Control (VC) breath type.

A ventilation “mode”, on the other hand, is a set of rules controlling how multiple subsequent breaths should be delivered. Modes may be mandatory, that is controlled by the ventilator, or spontaneous, that is that allow a breath to be delivered or controlled upon detection of a patient's effort to inhale, exhale or both. For example, a simple mandatory mode of ventilation is to deliver one breath of a specified mandatory breath type at a clinician-selected respiratory rate (e.g., one breath every 6 seconds). Until the mode is changed, ventilators will continue to provide breaths of the specified breath type as dictated by the rules defining the mode. A combination of mandatory and spontaneous breath types may also be delivered in a ventilation mode based on either detecting patient inspiratory effort or on a set respiratory frequency.

Different modes require a clinician to specify different parameters. For example, a mode employs mandatory breaths, the clinician may be required to select a mandatory breath type and specify a respiratory frequency. If a mode employs spontaneous breaths, the clinician may be required to select a spontaneous breath type as well as either a pressure trigger or a flow trigger. When a mode employs a combination of spontaneous and mandatory breath types, a clinician may be required to select some or all of the above parameters. Understanding ventilation modes is necessary to understand the different parameters that a clinician may have to set in conjunction with a given mode. Some ventilation modes are discussed below:

Assist/Control (A/C) Mode

When set to A/C mode, the ventilator may be set to deliver mandatory breaths to the patient. The patient may initiate breaths during a set period determined by various criteria including a respiratory frequency. The ventilator delivers patient initiated mandatory breaths when a spontaneous patient effort is detected. When a patient effort is not detected, the ventilator automatically delivers ventilator initiated mandatory breaths at the set respiratory frequency.

Synchronous Intermittent Mandatory Ventilation (SIMV) Mode

When set to SIMV mode, the ventilator may deliver mandatory breaths and spontaneous breaths. A SIMV breathing cycle may be determined based on the set respiratory frequency. The SIMV mode is set such that a patient initiated mandatory breath or a ventilator initiated mandatory breath is delivered in a mandatory interval during each SIMV breathing cycle. If patient effort is detected during the mandatory interval, the ventilator delivers a patient initiated mandatory breath and then transitions into a spontaneous interval for the remainder of the SIMV breathing cycle. On the other hand, if no patient effort is detected during the mandatory interval, the ventilator delivers a VIM breath at the end of the mandatory interval and then moves into the spontaneous interval for the rest of the SIMV breathing cycle.

Spontaneous (SPONT) Mode

When set to SPONT mode, the ventilator may deliver spontaneous breaths. When the ventilator detects patient effort, a spontaneous breath is delivered based on the selected spontaneous breath type.

Apnea Mode

A ventilator may be set to Apnea mode as a back-up ventilation mode to deliver mandatory breaths when the ventilator fails to detect spontaneous patient effort within a clinician-selected backup period. Specifically, the ventilator may be set to deliver breaths by one of the above described modes and may also be set to deliver backup ventilation in Apnea mode. That is, if spontaneous patient effort is not detected within the clinician-selected backup period, the ventilator delivers a series of mandatory breaths, the particular mandatory breath types depending on the ventilator settings. If sufficient patient effort is detected, spontaneous ventilation will resume per the previously selected mode and breath type.

The above ventilation modes provide an overview of exemplary ventilation modes. As will be appreciated, any number of ventilation modes known in the art are contemplated within the scope of the present application. As will be appreciated, before a ventilator administers any ventilation mode, various ventilation parameters must be input in associated with the ventilation mode. The present application provides a previous mode button that allows a clinician to administer any previous ventilation mode per the ventilation parameters stored in association with that ventilation mode. By using previously stored ventilation parameters, the clinician is spared the time that would be used re-inputting ventilation parameters for a previously administered ventilation mode. This spared time may allow the clinician to focus on other aspects of patient ventilation rather than re-determining which ventilation parameters are appropriate for a given patient.

Although the techniques introduced above and discussed in detail below may be implemented for a variety of medical devices, the present disclosure will discuss the implementation of these techniques for use in a mechanical ventilator system. The reader will understand that the technology described in the context of a ventilator system could be adapted for use with other therapeutic equipment having user interfaces, including graphical user interfaces (GUIs), for prompt startup of a therapeutic treatment.

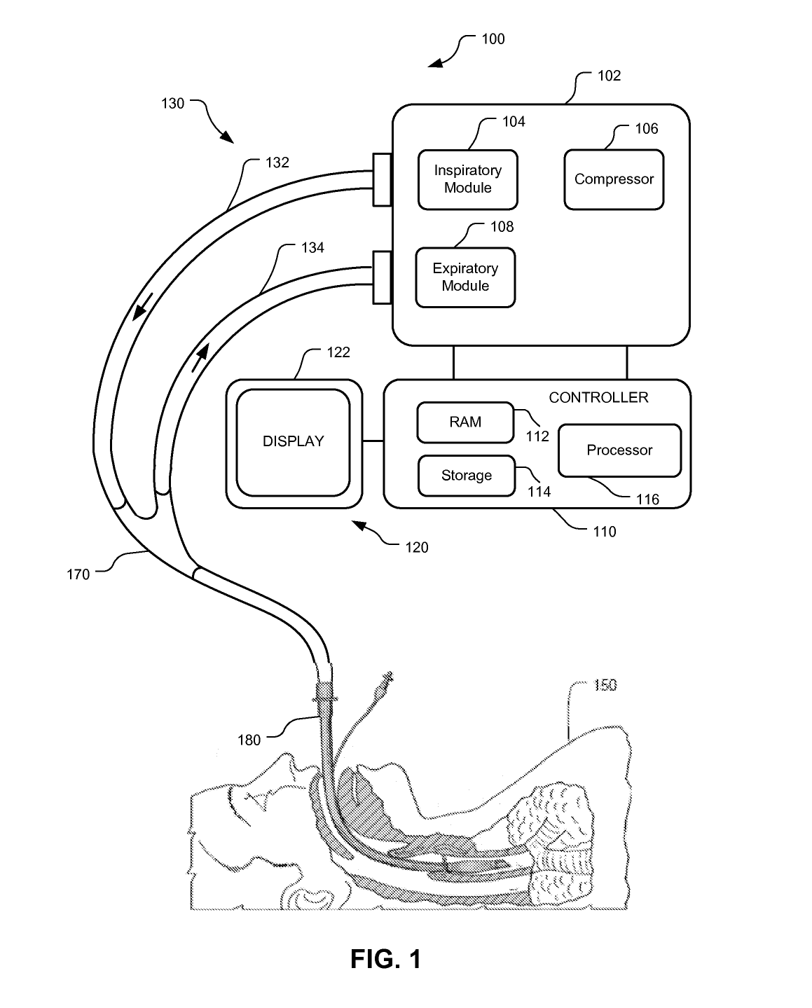

FIG. 1 is a diagram illustrating an embodiment of an exemplary ventilator 100 connected to a human patient 150. Ventilator 100 includes a pneumatic system 102 (also referred to as a pressure generating system 102) for circulating breathing gases to and from patient 150 via the ventilation tubing system 130, which couples the patient to the pneumatic system via an invasive (e.g., endotracheal tube, as shown) or a non-invasive (e.g., nasal mask) patient interface.

Ventilation tubing system 130 may be a two-limb (shown) or a one-limb circuit for carrying gases to and from the patient 150. In a two-limb embodiment, a fitting, typically referred to as a “wye-fitting” 170, may be provided to couple a patient interface 180 (as shown, an endotracheal tube) to an inspiratory limb 132 and an expiratory limb 134 of the ventilation tubing system 130.

Pneumatic system 102 may be configured in a variety of ways. In the present example, system 102 includes an expiratory module 108 coupled with the expiratory limb 134 and an inspiratory module 104 coupled with the inspiratory limb 132. Compressor 106 or other source(s) of pressurized gases (e.g., air, oxygen, and/or helium) is coupled with inspiratory module 104 to provide a gas source for ventilatory support via inspiratory limb 132.

The pneumatic system 102 may include a variety of other components, including mixing modules, valves, sensors, tubing, accumulators, filters, etc. Controller 110 is operatively coupled with pneumatic system 102, signal measurement and acquisition systems, and an operator interface 120 that may enable an operator to interact with the ventilator 100 (e.g., change ventilator settings, select operational modes, view monitored parameters, etc.). Controller 110 may include memory 112, one or more processors 116, storage 114, and/or other components of the type commonly found in command and control computing devices. In the depicted example, operator interface 120 includes a display 122 that may be touch-sensitive and/or voice-activated, enabling the display to serve both as an input and output device.

The memory 112 includes non-transitory, computer-readable storage media that stores software that is executed by the processor 116 and which controls the operation of the ventilator 100. In an embodiment, the memory 112 includes one or more solid-state storage devices such as flash memory chips. In an alternative embodiment, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

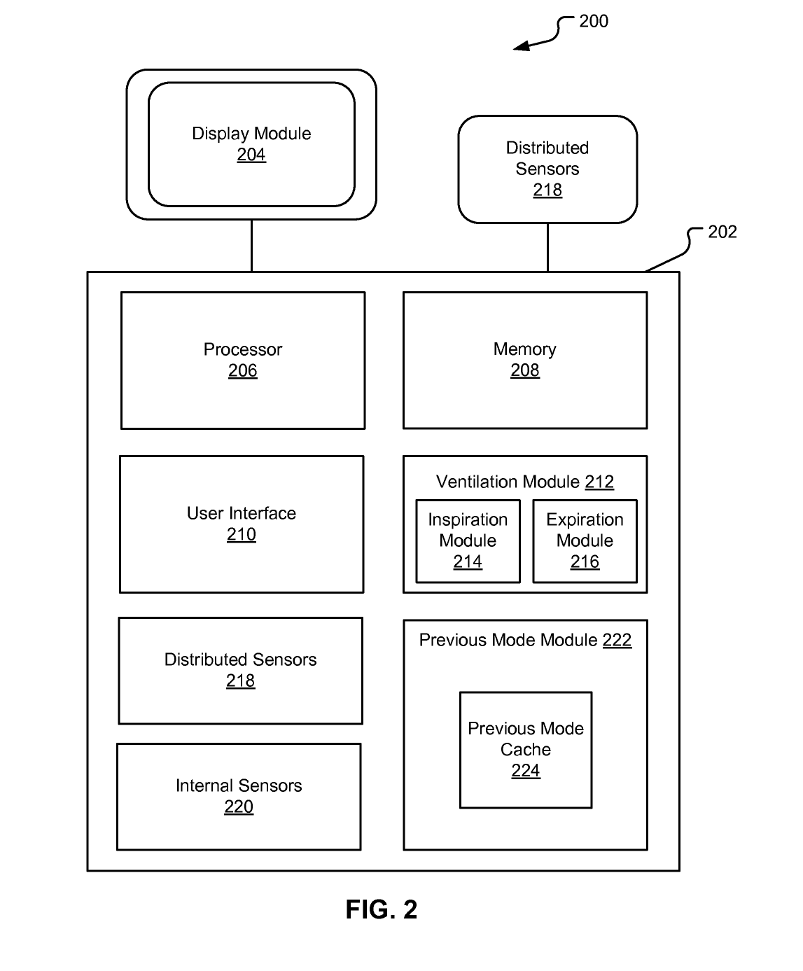

FIG. 2 is a block-diagram illustrating an embodiment of a ventilatory system for storing multiple previous modes.

Ventilatory system 200 includes ventilator 202 with its various modules and components. That is, ventilator 202 may further include, inter cilia, memory 208, one or more processors 206, user interface 210, ventilation module 212 (which may further include an inspiration module 214 and an expiration module 216), and previous stored modes module 222. Memory 208 is defined as described above for memory 112. Similarly, the one or more processors 206 are defined as described above for one or more processors 116. Processors 206 may further be configured with a clock whereby elapsed time may be monitored by the system 200.

The ventilatory system 200 may also include a display module 204 communicatively coupled to ventilator 202. Display module 204 provides various input screens, for receiving clinician input, and various display screens, for presenting useful information to the clinician. The display module 204 is configured to communicate with user interface 210 and may include a graphical user interface (GUI). The GUI may be an interactive display, e.g., a touch-sensitive screen or otherwise, and may provide various windows and elements for receiving input and interface command operations. Alternatively, other suitable means of communication with the ventilator 202 may be provided, for instance by a wheel, keyboard, mouse, or other suitable interactive device. Thus, user interface 210 may accept commands and input through display module 204. Display module 204 may also provide useful information in the form of various ventilatory data regarding the physical condition of a patient and/or a prescribed respiratory treatment. The useful information may be derived by the ventilator 202, based on data collected, and the useful information may be displayed to the clinician in the form of graphs, wave representations, pie graphs, or other suitable forms of graphic display. For example, a settings screen may be displayed on the GUI and/or display module 204 to configure hybrid mode ventilation.

Ventilation module 212 may further include an inspiration module 214 configured to deliver gases to the patient according to prescribed ventilatory settings. Specifically, inspiration module 214 may correspond to the inspiratory module 104 or may be otherwise coupled to source(s) of pressurized gases (e.g., air, oxygen, and/or helium), and may deliver gases to the patient. Inspiration module 214 may be configured to provide ventilation according to various ventilatory breath types per a selected ventilator mode. As discussed above, these breath types may include. Thus, the ventilation module 212 includes the algorithms and computer-readable instructions necessary to provide any desired breath type.

Ventilation module 212 may further include an expiration module 216 configured to release gases from the patient's lungs according to prescribed ventilatory settings. Specifically, expiration module 216 may correspond to expiratory module 108 or may otherwise be associated with and/or controlling an expiratory valve for releasing gases from the patient. By way of general overview, a ventilator may initiate expiration based on lapse of an inspiratory time setting or other cycling criteria set by the clinician or derived from ventilator settings (e.g., detecting delivery of prescribed tidal volume or prescribed pressure). Upon initiating the expiratory phase, expiration nodule 216 may allow the patient to exhale by opening an expiratory valve. As such, expiration is passive, and the direction of airflow is governed by the pressure gradient between the patient's lungs (higher pressure) and the ambient surface pressure (lower pressure). Although expiratory flow is passive, it may be regulated by the ventilator based on the size of the expiratory valve opening.

According to some embodiments, the inspiration module 214 and/or the expiration module 216 may be configured to synchronize ventilation with a spontaneously-breathing, or triggering, patient. Specifically, the ventilator may detect patient effort via a pressure-monitoring method, a flow-monitoring method, direct or indirect measurement of nerve impulses, or any other suitable method. Sensing devices may be either internal or distributed and may include any suitable sensing device, as described further herein. In addition, the sensitivity of the ventilator to changes in pressure and/or flow may be adjusted such that the ventilator may properly detect the patient effort, i.e., the lower the pressure or flow change setting the more sensitive the ventilator may be to patient triggering.

According to embodiments, a pressure-triggering method may involve the ventilator monitoring the circuit pressure, as described above, and detecting a slight drop in circuit pressure. The slight drop in circuit pressure may indicate that the patient's respiratory muscles are creating a slight negative pressure gradient between the patient's lungs and the airway opening in an effort to inspire. The ventilator may interpret the slight drop in circuit pressure as patient effort and may consequently initiate inspiration by delivering respiratory gases.

Alternatively, the ventilator may detect a flow-triggered event. Specifically, the ventilator may monitor the circuit flow, as described above. If the ventilator detects a slight drop in flow during exhalation, this may indicate, again, that the patient is attempting to inspire. In this case, the ventilator is detecting a drop in bias flow (or baseline flow) attributable to a slight redirection of gases into the patient's lungs (in response to a slightly negative pressure gradient as discussed above). Bias flow refers to a constant flow existing in the circuit during exhalation that enables the ventilator to detect expiratory flow changes and patient triggering. For example, while gases are generally flowing out of the patient's lungs during expiration, a drop in flow may occur as some gas is redirected and flows into the lungs in response to the slightly negative pressure gradient between the patient's lungs and the body's surface. Thus, when the ventilator detects a slight drop in flow below the bias flow by a predetermined threshold amount (e.g., 2 L/min below bias flow), it may interpret the drop as a patient trigger and may consequently initiate inspiration by delivering respiratory gases.

The ventilatory system 200 may also include one or more distributed sensors 218 communicatively coupled to ventilator 202. Distributed sensors 218 may communicate with various components of ventilator 202, e.g., ventilation module 212, internal sensors 220, and any other suitable components and/or modules. Distributed sensors 218 may detect changes in patient measurements indicative of crossing a Hybrid Mode threshold, for example. Distributed sensors 218 may be placed in any suitable location, e.g., within the ventilatory circuitry or other devices communicatively coupled to the ventilator. For example, sensors may be affixed to the ventilatory tubing or may be imbedded in the tubing itself. According to some embodiments, sensors may be provided at or near the lungs (or diaphragm) for detecting a pressure in the lungs. Additionally or alternatively, sensors may be affixed or imbedded in or near wye-fitting 170 and/or patient interface 180, as described above.

Distributed sensors 218 may further include pressure transducers that may detect changes in circuit pressure (e.g., electromechanical transducers including piezoelectric, variable capacitance, or strain gauge). Distributed sensors 218 may further include various flow sensors for detecting airflow (e.g., differential pressure pneumotachometers). For example, some flow sensors may use obstructions to create a pressure decrease corresponding to the flow across the device (e.g., differential pressure pneumotachometers) and other flow sensors may use turbines such that flow may be determined based on the rate of turbine rotation (e.g., turbine flow sensors). Alternatively, sensors may utilize optical or ultrasound techniques for measuring changes in ventilatory parameters. A patient's blood parameters or concentrations of expired gases may also be monitored by sensors to detect physiological changes that may be used as indicators to study physiological effects of ventilation, wherein the results of such studies may be used for diagnostic or therapeutic purposes. Indeed, any distributed sensory device useful for monitoring changes in measurable parameters during ventilatory treatment may be employed in accordance with embodiments described herein.

Ventilator 202 may further include one or more internal sensors 220. Similar to distributed sensors 218, internal sensors 220 may communicate with various components of ventilator 202, e.g., ventilation module 212, internal sensors 220, and any other suitable components and/or modules. Internal sensors 220 may employ any suitable sensory or derivative technique for monitoring one or more parameters associated with the ventilation of a patient. However, the one or more internal sensors 220 may be placed in any suitable internal location, such as, within the ventilatory circuitry or within components or modules of ventilator 202. For example, sensors may be coupled to the inspiratory and/or expiratory modules for detecting changes in, for example, circuit pressure and/or flow. Specifically, internal sensors may include pressure transducers and flow sensors for measuring changes in circuit pressure and airflow. Additionally or alternatively, internal sensors may utilize optical or ultrasound techniques for measuring changes in ventilatory parameters. For example, a patient's expired gases may be monitored by internal sensors to detect physiologic changes indicative of the patient's condition and/or treatment. Indeed, internal sensors may employ any suitable mechanism for monitoring parameters of interest in accordance with embodiments described herein.

As should be appreciated, ventilatory parameters are highly interrelated and, according to embodiments, may be either directly or indirectly monitored. That is, parameters may be directly monitored by one or more sensors, as described above, or may be indirectly monitored by derivation.

Ventilator 200 may further include previous mode module 222. Previous mode module may be communicatively coupled with ventilation module 212. When a ventilation mode is administered via ventilation module 212, the ventilation module may communicate ventilation parameters associated with the ventilation mode to previous mode module 222. Upon receipt of the ventilation parameters, the previous mode module may store the ventilation mode and associated ventilation parameters in a previous mode cache 224. In one embodiment, a previous mode cache 224 may be associated with a particular patient such that ventilator 200 may have multiple previous mode caches. Alternatively, previous mode cache 224 may be associated with multiple patients. In this embodiment, when previous mode module 222 receives information from the ventilator module 212 that a ventilation mode is being administered to a patient, the associated ventilation parameters and ventilation mode may be associated with an identifier corresponding to that patient. The previous mode cache 224 may also be accessed by the previous mode module when a request is received to retrieve ventilation parameters associated with a previously administered mode. The request may include an identifier associated with the patient. The previous mode cache 224 may then retrieve the ventilation parameters for the ventilation mode for the patient associated with the identifier. These ventilation parameters for the previously administered mode may then be communicated to ventilator module 212.

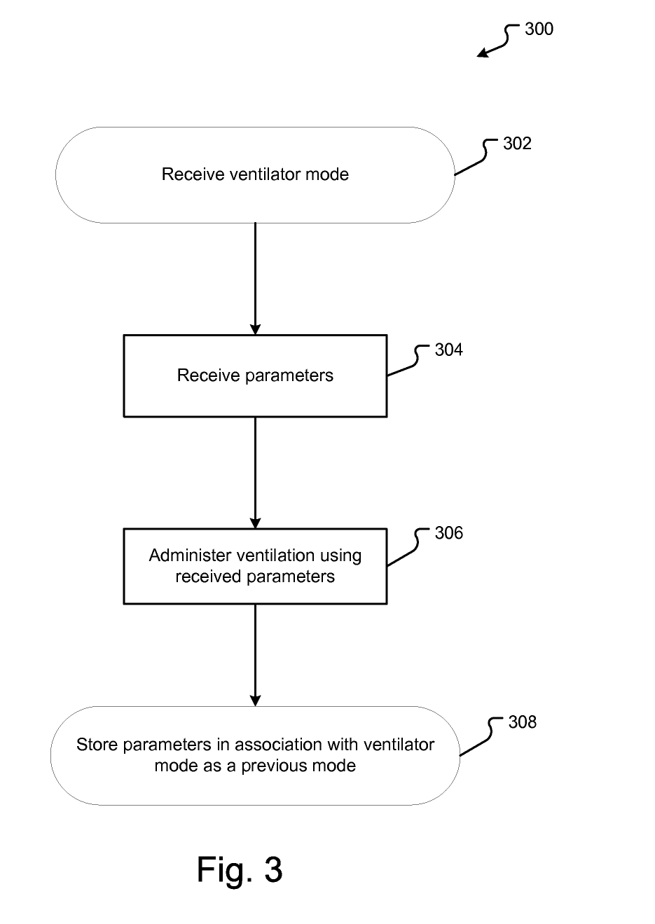

FIG. 3 is an illustrative method 300 for storing parameters for multiple ventilation modes.

At receive operation 302, a selection of a ventilation mode is received. As discussed above, different ventilation modes are appropriate in different ventilation situations. As such, the clinician selects the most appropriate ventilation mode for a patient and the patient's condition. Once a ventilation mode has been selected, flow proceeds to receive operation 304.

At receive operation 304, a selection of one or more ventilation parameters are received. Which ventilation parameters are received depends on which ventilation mode is selected. For example, if AIC mode is selected, selections for a mandatory breath type and respiratory frequency may be received. Furthermore, per the selected mandatory breath type, the A/C mode may also require a selection of other ventilation parameters. Alternatively, if SPONT mode is selected, selections for a spontaneous breath type and trigger type may be received. Furthermore, per the selected spontaneous breath type, the SPONT mode may also require a selection of other ventilation parameters. Once the required ventilation parameters are selected, flow proceeds to administer operation 306.

At administer operation 306, the selected ventilation mode is administered per the selected parameters. When the ventilation mode is administered, the ventilation mode and its associated parameters are stored as a previous mode in a previous mode cache. Flow then terminates.

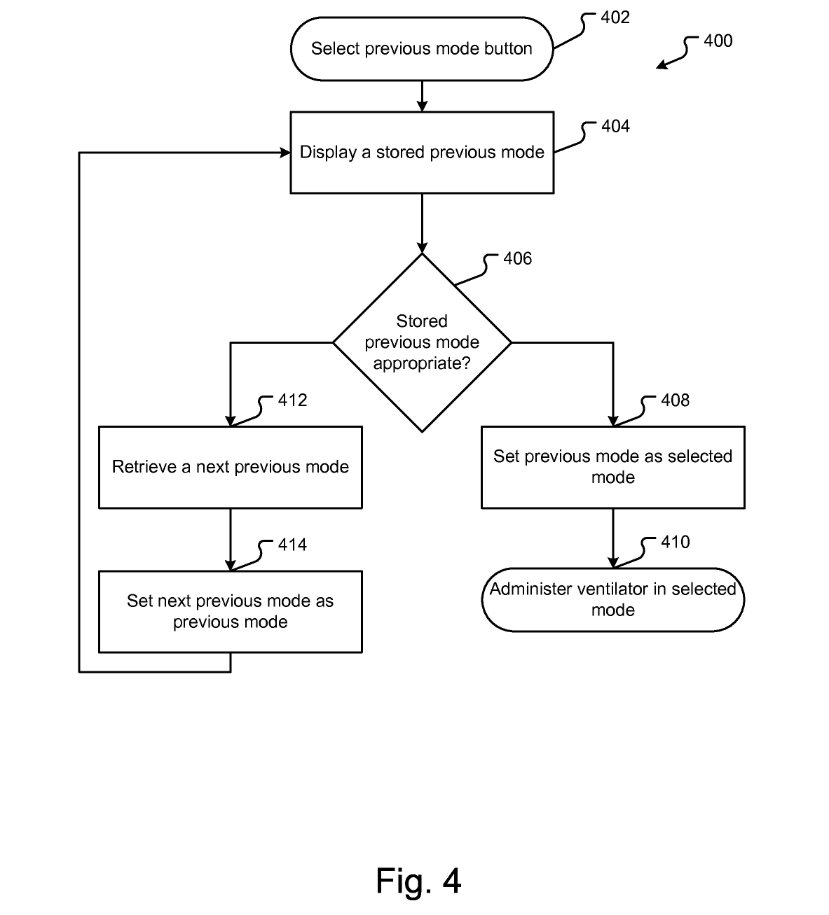

FIG. 4 is an illustrative method 400 for selecting a ventilation mode with previously stored parameters.

At select operation 402, a previous mode button is selected. Selection of a previous mode button may comprise physically contacting a previous mode button, selecting the previous mode button with a mouse, or any other mode of selection known in the art. Once the previous mode button is selected, flow proceeds to operation 404.

At display operation 404, a previous mode is displayed. In one embodiment, the previous mode is the last ventilation mode previously administered by the ventilator. Display of the previous mode may comprise displaying the name of the ventilation mode and associated stored ventilation parameters. In one embodiment, the ventilation mode name and stored ventilation parameters may be displayed on a graphical user interface. In another embodiment, the ventilation parameters associated with the displayed previous mode may be altered when the previous mode is displayed. In another embodiment the previous mode may be deleted from the previous mode cache. Once the previous mode is displayed, flow proceeds to determine operation 406.

At determine operation 406, a determination is made if the displayed previous mode is the appropriate mode for ventilation. In one embodiment, a determination may be made that the displayed previous mode is the appropriate ventilation mode for ventilation if an indication is received from the user that the displayed previous mode is appropriate. Such an indication may include selection of a set button on the graphical user interface. In another embodiment, such an indication may include receiving a second selection of the previous mode button, where the previous mode button is held for a certain period of time. For example, if the previous mode button is held for two seconds, an indication may be received that the displayed next previous mode is the appropriate ventilation mode. Alternatively, a determination may be made that the displayed previous mode is not the appropriate mode for ventilation if an indication is received that the displayed next previous mode is not appropriate. In one embodiment, such an indication may include selection of the previous mode button, where the previous mode button is not held for a period of time. If a determination is made that the displayed previous mode is the appropriate ventilation mode, flow proceeds to set operation 408. If a determination is made that the displayed previous mode is not the appropriate ventilation mode, flow proceeds to retrieve operation 412.

At set operation 408, the displayed previous mode is set as the selected ventilation mode. Once the displayed previous mode is set as the selected mode, flow proceeds to administer operation 410.

At administer operation 410, the ventilator administers the selected ventilation mode to the patient per the stored associated ventilation parameters. Once the selected ventilation mode is administered, flow terminates.

Alternatively, if a determination was made at determine operation 406 that the previous mode is not the appropriate ventilation mode, a next previous mode is retrieved at operation 412. In one embodiment, the next previous mode may be retrieved from a previous mode cache. As described above, the previous mode cache May store ventilation parameters associated with previously ventilation modes for a given patient. The previous mode cache may store any number of previously administered modes. Once a next previous mode is retrieved, flow proceeds to set operation 414.

At set operation 414, the retrieved next previous mode is set as the previous mode. Once the next previous mode is set as the previous mode, flow proceeds to display operation 404.

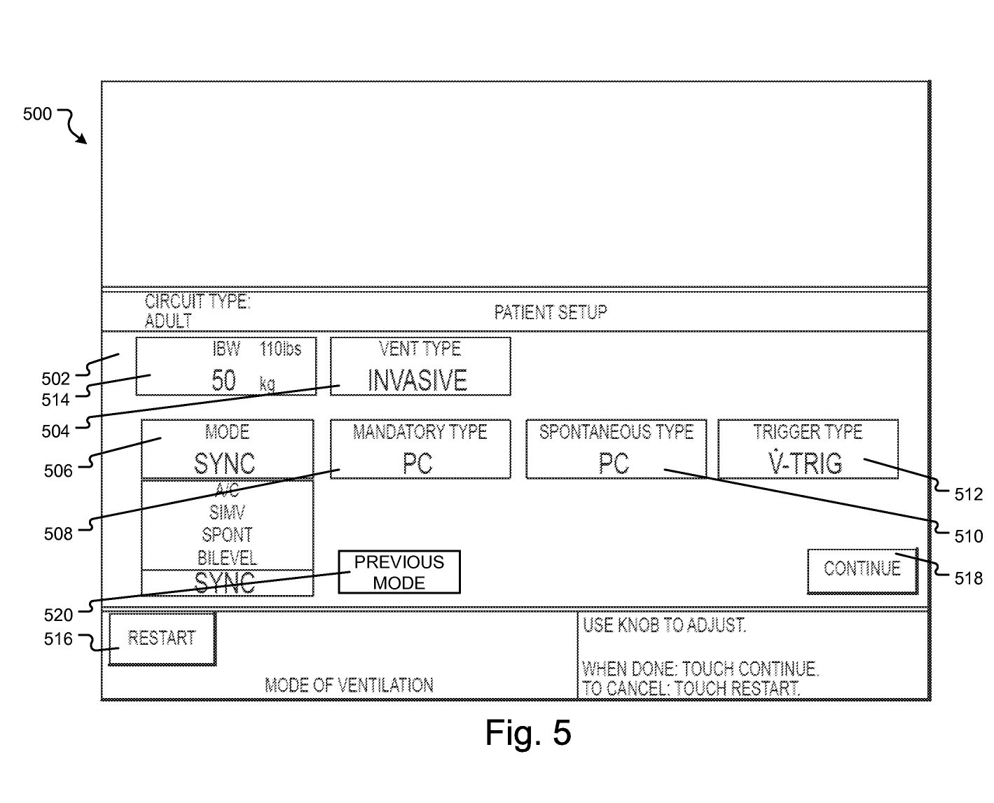

FIG. 5 depicts a patient set up interface 500 that includes a previous mode button for administering the ventilator

According to one embodiment, as illustrated by FIG. 4, patient setup interface 500 may include patient setup window 502. Patient setup window 502 may include one or more selectable elements to configure patient setup. Patient setup window 502 may include a Vent Type button 504. Vent Type button 504 allows a clinician to select a type of ventilation for the patient. In one embodiment, when the clinician selects the Vent Type button 504 a pull down menu appears underneath the Vent Type button 504 displaying vent type options (not depicted). The clinician can then select one of the vent type options to set as the Vent Type. The vent type options may include invasive and non-invasive. These vent type options correspond to the way that the patient was attached to the ventilator as discussed in detail with reference to FIG. 1. As will be appreciated, when a vent type option is selected, it is displayed in the Vent Type button 504 as depicted in patient setup window 502.

Patient setup window 502 may be further configured to include a Mode button 456. Like the Vent Type button 504, when a clinician selects the Mode button 506, a pull down menu appears under the Mode button 506. The pull down menu displays various modes options for selection. As will be appreciated, when a mode option is selected, it is displayed in the Mode button 506 as depicted in patient setup window 502.

The patient setup window 502 may be further configured to include a Mandatory Type button 508. In one embodiment, the Mandatory Type button 508 may only be displayed when the made displayed by Mode button 506 administers mandatory breaths. In another embodiment, the Mode button 506 may be displayed regardless however, when the mode displayed by Mode button 506, does not administer mandatory breaths, the Mandatory Type button 508 is filled with an indication that there is no mandatory type. For example the Mandatory Type button 508 may include an indication that there is no mandatory type such as a blank, the word “none”, “not applicable”, or any indication that there is no mandatory type. When the clinician selects the Mandatory Type button 508 a pull down menu appears under the Mandatory Type button 508. The pull down menu displays various mandatory type options for selection. The mandatory type options are mandatory breath types. As will be appreciated, when a mandatory type option is selected, it is displayed in the Mandatory Type button 508 as depicted in patient setup window 502.

The patient setup window 502 may be further configured to include a Spontaneous Type button 510. In one embodiment, the Spontaneous Type button 510 may only be displayed when the mode displayed by Mode button 506 administers spontaneous breaths. In another embodiment, the Mode button 506 may be displayed regardless however, when the mode displayed by Mode button 506, does not administer spontaneous breaths, the Spontaneous Type button 510 is filled with an indication that there is no spontaneous type. For example the Spontaneous Type button 510 may include an indication that there is no mandatory type such as a blank, the word “none”, “not applicable”, or any indication that there is no mandatory type. When the clinician selects the Spontaneous Type button 510 a pull down menu appears under the Spontaneous Type button 510. The pull down menu displays various spontaneous type options for selection. As will be appreciated, when a spontaneous type option is selected, it is displayed in the Spontaneous Type button 510 as depicted in patient setup window 502.

The patient setup window 502 may be further configured to include a Trigger Type button 512. When the clinician selects the Trigger Type button 512 a pull down menu appears under the Trigger Type button 512. The pull down menu displays various trigger type options for selection. These trigger types may include a flow trigger and a pressure trigger. As will be appreciated, the selected trigger type determines the patient measurement(s) used to determine if a patient is spontaneously triggering. In one embodiment, the clinician can choose from any of available trigger types such as pressure, flow, volume, patient effort, etc. As will be appreciated, when a trigger type option is selected, it is displayed in the Trigger Type button 512 as depicted in patient setup window 502.

The patient setup window 502 may include various other selectable elements. For example, the window may include an Ideal Body Weight button 514 and a restart button 516. Like the other buttons discussed above with reference to FIG. 5, the Ideal Body Weight button 514 may be selected to change the Ideal Body Weight setting of a patient. The restart button 516 may also be selected to restart the ventilator.

Once a clinician is satisfied with the settings displayed on the new patient setup window 502, the clinician may select the continue button 516 to configure the ventilator with the displayed settings. When the continue button 516 is selected, the displayed parameters on the Mandatory Type button 508, Spontaneous Type button 510, and Trigger Type button 512 are saved in association with the mode displayed in the Mode type button 506 for the patient. The parameters and mode may be saved as a previous mode as discussed above with reference to FIGS. 2-4.

The patient setup window 502 may also include a previous mode button 520. As described above, the previous mode button 520 may be used to select a stored previous mode. When the previous mode button 520 is selected, the parameters associated with the previously stored mode may be displayed in the patient setup window 502. In another embodiment, the parameters associated with the previously stored mode may be displayed in a new window. If the previous mode and associated parameters are appropriate, a clinician may select continue button 516 to administer ventilation using the previous mode and parameters. If the previous mode is not appropriate, the clinician may again select the previous mode button 520. By selecting the previous mode button 520, another previously stored mode and parameters will be displayed to the clinician on either patient setup window 502 or a new window. A clinician may continue to select the previous mode button 520 and display previously stored modes until an appropriate mode is displayed. Upon display of an appropriate mode, the clinician may select continue button 516 to administer ventilation using the previous mode and parameters.

It will be clear that the systems and methods described herein are well adapted to attain the ends and advantages mentioned as well as those inherent therein. Those skilled in the art will recognize that the methods and systems within this specification may be implemented in many manners and as such is not to be limited by the foregoing exemplified embodiments and examples. In other words, functional elements being performed by a single or multiple components, in various combinations of hardware and software, and individual functions can be distributed among software applications at either the client or server level. In this regard, any number of the features of the different embodiments described herein may be combined into one single embodiment and alternative embodiments having fewer than or more than all of the features herein described are possible.

While various embodiments have been described for purposes of this disclosure, various changes and modifications may be made which are well within the scope of the present technology. Numerous other changes may be made which will readily suggest themselves to those skilled in the art and which are encompassed in the spirit of the disclosure and as defined in the appended claims.

I claim:

1. A method for administering ventilation by a mechanical ventilator using a previously stored ventilation mode, the method comprising:
receiving a selection of a previous mode button;
displaying a first stored previous mode and first ventilation parameters associated with the first stored previous mode;
determining the displayed first stored previous mode is not an appropriate mode;
retrieving a second stored previous mode; and
displaying the second stored previous mode and second ventilation parameters associated with the second stored previous mode.

2. The method of claim 1, wherein the first ventilation parameters further comprise mandatory breath type parameters.

3. The method of claim 1, wherein the first ventilation parameters further comprise spontaneous breath type parameters.

4. The method of claim 1, wherein the second ventilation parameters further comprise mandatory breath type parameters.

5. The method of claim 1, wherein the second ventilation parameters further comprise spontaneous breath type parameters.

6. The method of claim 1, wherein determining further comprises receiving an indication that the first stored previous mode is not appropriate.

7. The method of claim 1, wherein the indication further comprises a second selection of the previous mode button.

8. The method of claim 1, wherein retrieving further comprises accessing a previous mode cache.

9. The method of claim 1, further comprising:
receiving an indication that ventilation should be administered using the second stored previous mode; and
administering ventilation in the second stored previous mode using the associated second ventilation parameters.

10. A ventilatory system for administering ventilation by a mechanical ventilator using a previously stored ventilation mode:
at least one processor; and
at least one memory, communicatively coupled to the at least one processor and containing instructions for administering ventilation using a previously stored ventilation mode that, when executed by the at least one processor, perform a method comprising:
receiving a selection of a previous mode button;
displaying a first stored previous mode and first ventilation parameters associated with the first stored previous mode;
determining the displayed first stored previous mode is not an appropriate mode;
retrieving a second stored previous mode; and
displaying the second stored previous mode and second ventilation parameters associated with the second stored previous mode.

11. The ventilatory system of claim 10, wherein the first ventilation parameters further comprise mandatory breath type parameters.

12. The ventilatory system of claim 10, wherein the first ventilation parameters further comprise spontaneous breath type parameters.

13. The ventilatory system of claim 10, wherein the second ventilation parameters further comprise mandatory breath type parameters.

14. The ventilatory system of claim 10, wherein the second ventilation parameters further comprise spontaneous breath type parameters.

15. The ventilatory system of claim 10, wherein determining further comprises receiving an indication that the first stored previous mode is not appropriate.

16. The ventilatory system of claim 10, wherein the indication further comprises a second selection of the previous mode button.

17. The ventilatory system of claim 10, wherein retrieving further comprises accessing a previous mode cache.

18. The ventilatory system of claim 10, further comprising:
receiving an indication that ventilation should be administered using the second stored previous mode; and
administering ventilation in the second stored previous mode using the associated second ventilation parameters.

19. A graphical user interface for administering ventilation by a mechanical ventilator using a previously stored ventilation mode, the ventilator configured with a computer having a user interface including the graphical user interface for accepting commands, the graphical user interface comprising:
at least one window associated with the graphical user interface;
one or more elements within the at least one window, comprising at least one of:
a previous mode button allowing the selection of any one ventilation mode of the previously administered ventilation modes and the ventilation parameters associated with the one ventilation mode.

20. The graphical user interface of claim 19, further comprising:
a continue button that, when selected, indicates that the currently displayed ventilation mode and its associated currently displayed ventilation parameters should be stored as a previous mode.
